(19) United States
(12) Patent Application Publication
(10) Pub. No.: US 2022/0071747 A1
(43) Pub. Date: Mar. 10, 2022

(54) TREATMENT SYSTEM FOR CLEANING A COMPONENT CONTAMINATED BY BIOFILM, IN PARTICULAR AN IMPLANT PART

(71) Applicant: Markus SCHLEE, Forchheim (DE)

(72) Inventor: Markus SCHLEE, Forchheim (DE)

(21) Appl. No.: 17/530,884

(22) Filed: Nov. 19, 2021

Related U.S. Application Data
(63) Continuation of application No. 16/462,010, filed on May 17, 2019.

(30) Foreign Application Priority Data
Nov. 18, 2016 (EP) 16199650.9

(51) Int. Cl. A61C 17/00; A61C 8/00; A61C 19/00; A61L 2/03

(52) U.S. Cl. CPC A61C 17/00; A61C 8/0087; A61L 2/0011; A61L 2/03; A61C 8/0089; A61C 19/002

(57) ABSTRACT

The invention relates to a treatment system (I) for cleaning a component contaminated by biofilm, more particularly for cleaning bacterially contaminated surfaces of bone implants or dental implants (30), the treatment system comprising at least two conductor elements (2, 4) connected to an electrical supply unit (10) to form an electrical circuit. One of the conductor elements can be brought into electrical contact with the component in need of treatment. The object of the invention is to provide a treatment system of this type which allows a particularly acceptable and flexible treatment. To this end, according to the invention at least one of the conductor elements (2, 4) is designed as a diamond electrode (26).

### CROSS-REFERENCES TO RELATED APPLICATIONS

This application is a continuation and claims benefit of U.S. patent application Ser. No. 16/462,010 filed May 17, 2019, which is a 371 and claims benefit of PCT Application No. PCT/EP2017/079583 filed Nov. 17, 2017, which claims benefit of European Patent Application No. 16199650.9 filed Nov. 18, 2016, the specification(s) of which is/are incorporated herein in their entirety by reference.

The invention relates to a treatment system for cleaning a component contaminated with biofilm, in particular for cleaning bacterially contaminated surfaces of bone implants or dental implants, with at least two conductor elements connected to an electrical supply unit for forming an electrical circuit, one of which can be brought into electrical contact with the component in need of treatment.

Such a treatment system is known from WO 2014/122188 A1, whose disclosure is incorporated to its full extent by reference. This treatment system is provided in particular for use with an implant part and for cleaning a dental-implant part. Such a cleaning of an implant part may be desirable or necessary to guarantee or promote the growing-in or preservation of the implant which shall be inserted or has been inserted into the bone substance. In fact, a biofilm may form on the firm surface of implants, enclosed by tissue and tissue liquid, which biofilm is colonized by bacteria which may finally lead to chronic and recurrent infections. This syndrome is called periimplantitis. In particular in the dental area, similar to parodontitis, a combination of neglected mouth hygiene, adhesion of a biofilm on the usually microrough surface of the dental implant, and other factors lead to the full picture of periimplantitis, which is characterized by an increasing loading and destruction of the hard and soft tissues. The areas where the hard and/or soft tissues retreat are usually covered by a biofilm.

The cleaning method described in the above-mentioned application is based on the concept to kill and/or remove the biofilm or the germs forming the contamination, starting from the implant surface, without damaging the implant surface. For this purpose, an electrolytic process is provided, by which the ions (cations and/or anions) are conveyed by means of electrostatic forces through the biofilm. These ions react chemically or electrochemically on the implant surface. Through these reactions, new compositions of matter are created and/or the ions themselves and/or parts of these ions are converted into the atomic state. Furthermore, it is also possible that the ions react with the surface material (e.g. development of an oxide layer or erosion of material).

The germicidal effect of this process is based on different effects. On the one hand, ions from the biofilm itself (and also from the bacteria) are transported to the anode or cathode through the application of an electric voltage. This may lead to a killing of bacteria and viruses. Furthermore, the ions, while passing through the biofilm, may undergo biochemical reactions, which may also lead to a killing of bacteria and/or viruses. Another possibility of killing consists in that the compositions of matter newly formed on the implant surface possess an antibacterial and/or antiviral and/or antifungal effect. This may, of course, also happen when the ions are converted into the atomic state.

The treatment system described in the above-mentioned patent application is designed for a particularly simple handling and a particularly flexible use, without requiring that the prosthetics are disassembled if an implant is contaminated only relatively slightly by biofilm. To this end, the known system comprises two conductor elements configured in the manner of electrodes, one of which can be brought into direct electrical contact with the component in need of treatment, the other one forming a media cannula for introducing the ion-holding fluid into the environment of the component in need of treatment and, at the same time, making use of the electrical conductibility of the treatment fluid, forming the second connecting line to the common electrical supply unit which is necessary for forming the closed circuit.

The ions required according to the concept for the use of the treatment system known from the above-mentioned patent application are provided in that case by providing a suitable ion-holding treatment fluid and feeding it via the media cannula to the spatial region to be treated. Although the treatment fluid is suitably chosen and composed with regard to its content of ions (supplied, for example, by corresponding salts or acid fractions) in view of the requirements of acceptability, effects on the human tissue and the like, certain ion components are necessary in view of the intended treatment success and the effectiveness of killing germs, which might be detrimental with regard to the acceptability and biocompatibility. Furthermore, the reliable provision of the necessary treatment fluid might be costly.

The invention is, therefore, based on the problem to provide a treatment system of the above-mentioned type, with which the desired treatment success is possible without the necessity of feeding an ion-holding treatment fluid.

This problem is solved according to the invention by designing at least one of the conductor elements in the region of its free end as a diamond electrode.

Advantageous embodiments of the invention are the subject matter of the dependent claims. Further and/or alternative advantageous embodiments of the invention are also obvious from the description of the figures.

By “diamond electrode”, one understands in particular an electrode whose surface region, via which an interaction with the environment can take place, consists of diamond as a basic material, advantageously of boron-doped diamond. In particular, such a diamond electrode may consist of a metallic or ceramic base body, which is coated with diamond, because in general, diamond is suitable for use as a unique electrode material. If diamond is doped with few percent of boron, a semi-conductive diamond crystal is produced instead of the non-conductive diamond. If an overvoltage of few volts is applied on these diamond crystals, for example in a bipolar operating mode, the diamond surface possesses a particularly high oxidation power, without, however, being attacked or even destroyed itself. Boron-doped diamond offers a conductive surface for the production of short-lived hydroxyl radicals or OH⁻ ions. Upon contact with organic contaminations, these are oxidized directly to carbon dioxide (CO₂) and water (H₂O). Excessive hydroxyl radicals stabilise by reacting with water and AD soda to form oxidizing agents (active oxygen). The percarbonates and peroxides formed thereby provide a depot effect for the disinfection, whereby the formation of undesired micro-organisms is prevented or any germs input can be deactivated.

Accordingly, the use of diamond electrodes is known, for example, for the electrochemical water conditioning, as described in WO 2009/052163 A2.

It is, however, of particular importance for the now intended purpose in a treatment system of the above-mentioned type that, together with, and corresponding to, the hydroxyl radicals or OH⁻ ions, due to the decomposition of water molecules taking place on the diamond electrode, hydrogen ions or H⁺ ions are produced, which are particularly significant for the preferably intended treatment of a component contaminated with biofilm or other attached contaminations. When the component needing treatment is suitably wired as the cathode (or “negative pole”) of the circuit, the H⁺ ions produced on the diamond electrode migrate towards the component, penetrating the biofilm attached thereto. In particular because of their small size, the H⁺ ions relatively easily penetrate the biofilm or any hydrocarbons or the like attached to the surface and reach the cathode, i.e. the component in need of treatment as such. On the cathode, i.e. on the surface of the component in need of treatment, the H⁺ ions are reduced and take up an electron, so that molecular hydrogen (H₂) is formed. This molecular hydrogen is produced directly on the surface of the cathodically wired component and thus, under the attached biofilm or other attached contaminations, such as, for example, hydrocarbons. The hydrogen produced forms gas bubbles, which then rise and escape to the atmosphere. In doing so, these gas bubbles penetrate the biofilm or other attached contaminations, thus detaching them from the surface of the component and “carrying them along”. By means of a preferably provided continuous tracking by a flushing fluid, for example water, the detached contaminations are continuously diluted and washed away from the surface of the component to be treated, so that a reliable and sustained cleaning is achieved.

Through the now intended use of such a diamond electrode in the treatment system for cleaning a component contaminated with biofilm, it can, therefore, be achieved that the ions necessary for the combined treatment envisaged according to the concept, consisting of an application of electrical current on the one hand and a treatment with suitable ions on the other hand, do not necessarily have to be supplied externally and via an expensive supply system. Rather can these ions be directly generated on site during the process control and by the process itself, in the form of the H⁺ ions produced on the diamond electrode. That means that the treatment system modified in this manner can do without any appreciable supply of active ions; it just requires a process control in an aqueous environment or under supply of a hydrous or water-based treatment fluid, so that the decomposition of water molecules under formation of the hydroxyl radicals and the H⁺ ions can take place at the diamond electrode.

Each of the conductor elements is preferably connected with one pole of a power supply unit, so that an electric current path can be formed through a suitable handling and contacting with corresponding conductive elements. In a preferred embodiment, the conductor element(s) properly speaking are designed in the manner of needle-shaped electrodes, so that a user is able to locally position the respective free end, i.e. the end which is not connected via a connection cable with the power supply, of the needle-shaped electrode very precisely on a target spot and, thus, to adjust the current path with very high precision.

In an additional or alternative advantageous embodiment, that part of the respective conductor element which is configured as a diamond electrode is designed for a particularly large effective surface, so that a particularly large effective surface can be provided for the intended decomposition of the water molecules. To this end, the diamond electrode can be configured, for example, as a hollow cylinder, so that both the outer surface and the inner surface of the hollow cylinder are available as an effective surface. Alternatively or additionally, the surface of the diamond electrode can also be provided with a structure, for example a honeycomb or grid structure, and/or with an applied roughness, so that the effective surface is correspondingly enlarged, even with unchanged outer dimensions. Furthermore, the diamond electrode can preferably be positioned relatively near the free end of the respective conductor element or else can be configured as the latter's free contact tip, so that the intended process-related production of the H⁺ ions can take place relatively near the latter's intended place of use, i.e., for example, in the immediate vicinity of the biofilm to be treated.

In a preferred embodiment, the ion current path and the electron current path can be designed in one component, in order to simplify treatment and handling. In this case, they can be arranged one beside the other or one coaxially inside the other. The coaxial configuration can be designed such that the electron current path is executed inside the ion current path (or vice versa).

In general, due to the concept, the functional principle of the treatment system does not depend on the supply of a separate or special treatment fluid, because the ions necessary for the treatment are directly generated in the process control. However, a sufficient and advantageously continuous supply of a flushing means, preferably water or a fluid based on water, is advantageous for the process control and, therefore, preferred, so that, on the one hand, sufficient water reserves in the environment of the diamond electrode and thus the continuous production of the H⁺ ions is always guaranteed, and, on the other hand, the detached parts of the biofilm or of the contaminations can be removed reliably and continuously by the flushing-means flow.

Alternatively or additionally, a media cannula, for example in the form of a pipette or application lance, intended for yielding a treatment fluid, is advantageously provided. The treatment fluid applied is preferably a water-based or hydrous fluid, to provide sufficient water reserves for generating the hydroxyl radicals and H⁺ ions in the region of the diamond electrode. Advantageously, the inner space of the media cannula is connected with the electrical supply unit in an electrically conductive manner, so that the supplied treatment fluid can itself form the current path to the supply unit, in the region of the media cannula.

In a particularly preferred application, which is also considered as independently inventive, the diamond electrode and, with it, in particular also the above-explained treatment system, is used for cleaning a dental-implant part, in particular a dental implant also referred to as “post part”, a superstructure part also referred to as “abutment” for a dental implant, a crown or the like. In a preferred embodiment, treating or cleaning of the implant part can in that case be effected in the inserted condition of the implant, i.e. in particular in the patient's oral cavity. In an alternative, very particularly preferred use, the diamond electrode and, with it, in particular the above-explained treatment system, can, however, also be used outside the patient's mouth, for example in a treatment bath, for cleaning and possibly also disinfecting an implant part, in particular also brand-new components.

The surface of implants as regards its hydrophilicity is of particular importance for the growing-in behavior (the so-called osseointegration) into the bone tissue: examinations have shown that a hydrophilic surface promotes a good blood flow in the surrounding bone tissue, after insertion of an implant, which, in turn, promotes the osseointegration. Therefore, one tries to produce brand-new implants with a hydrophilic surface promotive of osseointegration. However, it has also turned out that implants prepared in such a way in the factory, degrade in the course of time with regard to their surface properties, in particular if they are stored too long or with insufficient care, so that an originally hydrophilic surface might become hydrophobic, thus greatly deteriorating the growing-in behavior of the implant. It has surprisingly turned out that the treatment according to the invention, now provided in a variant of the present invention, of an—otherwise brand-new—implant, prior to its insertion, with a diamond electrode and the above-described treatment concept can reliably restore the hydrophilicity of the surface. Therefore, it is particularly preferred to treat an implant, in particular a dental implant, with the above-mentioned treatment system prior to its insertion into the bone tissue.

In an alternative variant, which is also considered as independently inventive, a diamond electrode of the above-described type, in particular the treatment system of the above-described type, is used for disinfecting or even sterilizing a component, for example an implant intended for being inserted into the bone substance, or else, of osteosynthetic materials, such as, for example, plates or screws or also medical or therapeutical instruments or tools. Especially the measures for sterilizing the medical or surgical instruments prior to an operation or another intervention to be effected on the human body can be relatively expensive and entail long preparation times. It has most surprisingly turned out that such a sterilization of instruments can be carried out in a reliable manner and with extremely short treatment times in a treatment system of the above-mentioned type, which brings considerable advantages as to time and costs.

In another alternative variant, which is also considered as independently inventive, a diamond electrode of the above-described type is used for cleaning and/or conditioning propellers, rowing equipment and/or hulks of boats affected by fouling. Friction measurements have shown that the resistance of a boat in the water may increase by up to 50% due to fouling. That means an undesired increase of energy consumption and a reduction of the speed at which the boat or ship can sail. The phenomenon of pollution and fouling of boat hulks or other boat parts through algae, shells and other inorganic and organic parts depends on the water quality (freshwater, saltwater, pollution, overfertilization), temperature, incidence of light, and industrial contamination.

A protective coat of antifouling or other substances can usually not completely prevent fouling, and it gets detached in the course of time. Therefore, usually a manual cleaning is necessary, which is troublesome and expensive (diving, boat lift). Alternatively, sandblasting is also possible, which, however, also negatively influences the friction resistance of the propeller. Coatings are toxic and ecologically harmful.

According to the variant now provided, which is considered as an independent invention, all these depositions and appositions can be removed from metallically conductive surfaces (propellers, metallic ship hulks, rowing equipment, conductive plastic hulks, etc.) by means of the galvanoelectric method. For this purpose, the respective component, for example the propeller, is suitably connected with a voltage source (for example lead accumulator, wall socket and/or voltage supplier) and negatively charged. Furthermore, a diamond electrode is positioned at a certain distance. The diamond electrode decomposes, as described above, water molecules under the formation of hydroxyl radicals and H+ ions, and saltwater is an electrolyte anyhow, so that the conductibility required for the galvanic conditioning is given.

During the execution of the treatment, the produced H+ ions creep under the organic and inorganic depositions, under an alkalization which is insignificant for the process, and migrate to the anode, i.e. to the component to be treated (e.g. the propeller). The same will happen in case of a colonization with barnacles, sea mussels, etc. The latter are, in fact, fixed on the boat hulk by mussel glue or the like, but are in constant water exchange with their surroundings. The H+ ions recombine on the anode, taking up an electron, to form molecular hydrogen, and H₂ is produced. The latter forms little gas bubbles immediately in the surface region of the component, and these bubbles mechanically detach the depositions when escaping and rising to the water surface.

The method according to this independently inventive variant is simple, saves a dry-docking of the boats or ships and a mechanical cleaning, which must often be made by divers in a troublesome manner. To carry out this method, it is imaginable to apply the voltage with a clamp directly on the shaft of the ship's propeller, on ladders on the ship's sides or the like. Alternatively or additionally, a device might be already available in the boat, with which the shaft can be negatively charged, thus also negatively charging the ship's propeller.

The advantages achieved with the invention consist in particular in that, by using a diamond electrode in the treatment system, the ions required for the intended process control on the component or implant in need of treatment can be generated directly “on site” and in the immediate vicinity of the treatment zone properly speaking by transformation of water molecules on the diamond electrode, under release of hydroxyl radicals and H⁺ ions. The latter are a highly efficient cleaning means, which can be used for specifically detaching contaminations, biofilm or germs.

### BRIEF DESCRIPTION OF THE SEVERAL VIEWS OF THE DRAWING(S)

An exemplary embodiment of the invention is explained in detail by means of a drawing, in which

FIG. 1 shows a treatment system for cleaning a component contaminated with biofilm,

FIGS. 2a and 2b show two alternatives of a cleaning bath using the treatment system of FIG. 1,

FIG. 3is an enlarged detail of a treatment system according to FIG. 1,

FIG. 4 is an enlarged detail of an alternative embodiment of the treatment system of FIG. 1,

FIG. 5a is a longitudinal section of a detail of an alternative treatment system (FIG. 5a); FIG. 5b is a lateral view; and FIG. 5c is a cross-sectional view, and

FIG. 6 shows a sterilization system.

Identical parts are identified in all figures by the same reference numbers.

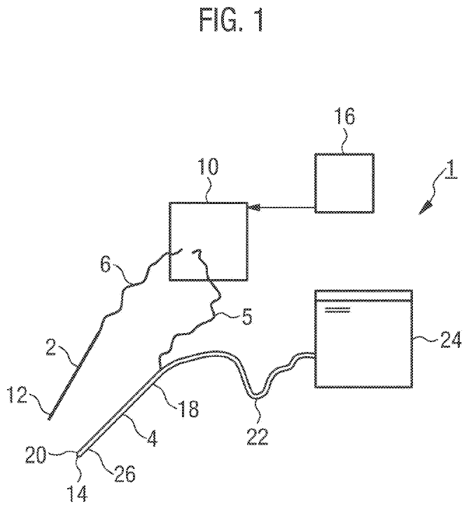

The treatment system 1 according to FIG. 1 is provided for cleaning a component contaminated with biofilm, in particular an implant part, and/or for sterilizing a component or instrument intended for use in or on the human body. The treatment system 1 is designed for an electrolytic cleaning concept, in which the component in need of treatment is charged with ions in a purposeful and localized manner and then a current flow through the component in need of treatment and possibly the treatment fluid is generated. To this end, the treatment system 1 comprises two conductor elements 2, 4, each of which is connected with an electrical supply unit 10 via a connection cable 6, 8, to form an electrical circuit.

The treatment system 1 of the exemplary embodiment is designed for yielding the current flow provided for the purpose of cleaning the component in need of treatment in a purposeful and localized manner in the spatial region needing treatment. To enable this, the conductor elements 2, 4 are designed in their free end regions in the manner of needle-shaped electrodes, so that a user is able to locally position the respective free end 12 or 14, i.e. the end which is not connected via the respective connection cable 6, 8 with the supply unit 10, of the needle-shaped electrode very precisely on a target spot and, thus, to adjust the current path with very high precision. The treatment system 1 is built up according to the design principle that the electric current is supplied to the component in need of treatment and the latter can be used as an electrode.

For this purpose, the conductor element 2 of the exemplary embodiment according to FIG. 1 is designed in the manner of a “conventional” electrode, i.e. in particular as an electrically conductive needle-shaped element made of metal, it can, however, also be made of any other conductive material. The conductor element 2 is provided on its outsides with an electric insulation and has only on its free end 12 an exposed metallic contact tip. In operation, the latter can suitably be pressed against the component in need of treatment, thus bringing about an electrical contact thereto. Alternatively, the conductor element 2 can, of course, also be clamped or screwed onto the component or suitably fastened thereto in another manner. The conductor element 2 is electrically connected with one of the poles of the electrical supply unit 10, in particular a current or voltage source.

A control unit 16, via which the current or voltage supplied can be controlled and set, is associated with the electrical supply unit 10.

Furthermore, the treatment system 1 comprises a media cannula 18, via which a treatment fluid can be conducted and output via an outlet opening 20. The media cannula 18 is integrated in the second conductor element 4 and, therefore, also has an elongated shape at its end region, so that a specifically localized and controlled output of the treatment fluid is possible. On the media side, the media cannula 18 is connected via a connection hose 22 with a storage container 24 for the treatment fluid. The control unit 16 additionally acts upon a conveyor system (not shown in detail) of the connection hose 22, with which the flow rate of the treatment fluid through the connection hose 22 can be adjusted. Alternatively, of course, the second conductor element 4 can also be designed in a constructionally separate manner and independently of the media cannula 18 and the connection hose 22.

The treatment system 1 is specifically designed for a combined treatment of the component in need of treatment with ions, on the one hand, and by energization, on the other hand. In order to be able to do without the supply of the ions provided for the treatment from an external source and, furthermore, to make the system particularly acceptable for an application in human tissue, the conductor element 4 is configured in the region of its free end 14 as a diamond electrode 26, in the exemplary embodiment, as a boron-doped diamond electrode 26. In case of use in an aqueous environment and in case of energization of this diamond electrode 26 with suitable parameters, for example in a bi-polar operating mode with an overvoltage of few volts, short-lived hydroxyl radicals or OH⁻ ions are generated. Upon contact with organic contaminations, these are oxidized directly to carbon dioxide (CO₂) and water (H₂O). Excessive hydroxyl radicals stabilise by reacting with water and AD soda to form oxidizing agents (active oxygen). The percarbonates and peroxides formed thereby provide a depot effect for the disinfection, whereby the formation of undesired microorganisms is prevented or any germs input can be deactivated.

Together with, and corresponding to, the hydroxyl radicals or OH⁻ ions, hydrogen ions or H⁺ ions are also produced, due to the decomposition of water molecules taking place on the diamond electrode 26, which can directly be used for ionic treatment of the component in need of treatment. When the component to be treated is suitably electrically connected via the conductor element 2 to the negative pole of the electrical supply unit 10, i.e. in case of cathodic wiring of the component to be treated, the H⁺ ions generated on the diamond electrode 26 migrate towards the component, penetrating the biofilm attached thereto or any contaminations, such as hydrocarbons, attached thereto. On the surface of the component in need of treatment, the H⁺ ions are reduced and take up an electron, so that molecular hydrogen (H₂) is formed. This molecular hydrogen is produced directly on the surface of the cathodically wired component and thus, under the attached biofilm or other attached contaminations, such as, for example, hydrocarbons. The hydrogen produced forms gas bubbles, which then rise and escape to the atmosphere. In doing so, these gas bubbles penetrate the biofilm or other attached contaminations, thus detaching them from the surface of the component and “carrying them along”.

Consequently, the treatment system 1 could also be used without the media cannula 18 and, thus, without the supply of a treatment fluid, if in the environment of the treatment process there is sufficient water to produce the above-mentioned radicals—for example in the patient's oral cavity during the treatment of a dental implant. The media cannula is, however, preferably provided, in order to guarantee in any case a sufficient supply of water as a base material for the production of ions and to guarantee in particular, in the manner of a flushing, the removal and continuous dilution of the detached contaminations. Consequently, a fluid based on water or having a sufficiently high content of water is preferably provided as a treatment fluid.

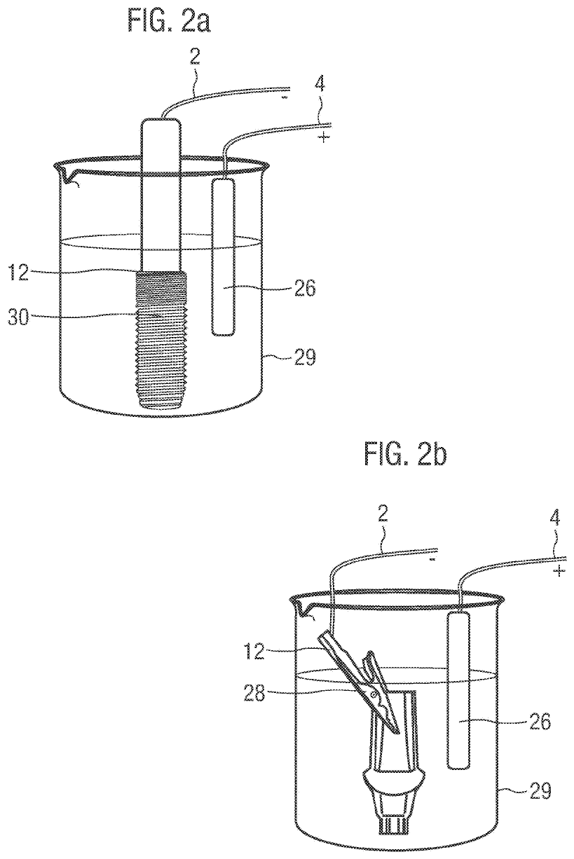

An exemplary embodiment of this variant in which the media cannula 18 is not absolutely necessary, is shown in FIG. 2 for two types of application, namely for the conditioning and/or sterilization of a brand-new dental implant 30 (FIG. 2a) and for the conditioning and/or sterilization of an abutment for a dental implant 30 (FIG. 2b). This use of the diamond electrode 26 for cleaning or sterilization purposes shown in FIG. 2, considered as independently inventive, is suited for a multitude of components and preferably, for example for all types of implants and their components, for surgical or other medical instruments, in particular immediately prior to their use in or on the human body, for gingiva formers and the like.

In the variant shown in FIG. 2, the use of the treatment system 1 is provided in the manner of a water bath. The component in need of treatment, for example the dental implant 30, is in this case connected to the free end 12 of the conductor element 2. This can be effected, as shown in FIG. 2b by way of example, via a clamp 28 connected to the end side of the conductor element 2. As shown in FIG. 2, the respective component, for example the dental implant 30, is immersed for treatment in a cleaning bath available in a container 29. The cleaning bath contains a suitably chosen treatment fluid based on water, so that the above-described generation of the hydroxyl radicals and H⁺ ions can take place. Furthermore, the diamond electrode 26 is immersed in the cleaning bath. Then, for the treatment, a current flow through the component, the cleaning bath and the diamond electrode 26 is adjusted by means of the supply unit 10, so that the radicals provided for cleaning and sterilization are generated in the above-described manner, decontaminating the component. Particularly advantageously, such a treatment is carried out on a brand-new dental implant 30 intended for being inserted, in order to reliably restore the hydrophilicity of the surface correspondingly prepared by the factory.

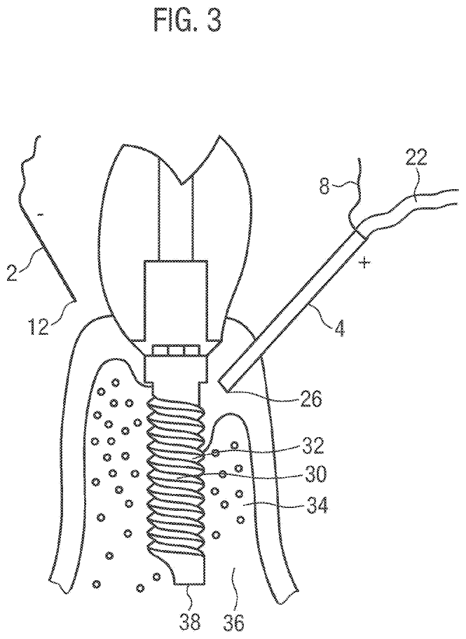

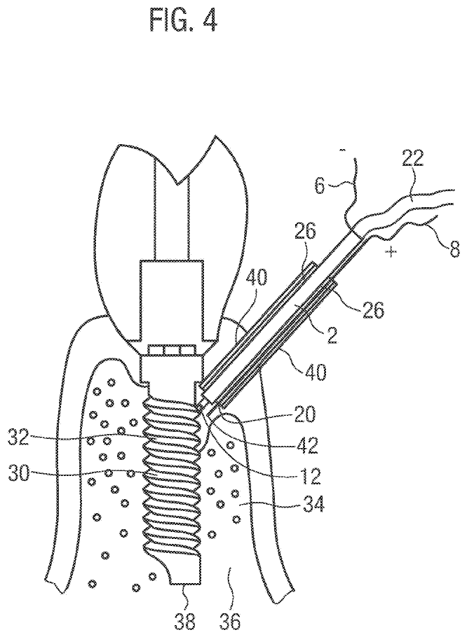

Alternatively, the treatment system 1 can, however, also be used for the treatment of a dental implant 30 already inserted into a patient's jaw bone, as is shown in the exemplary embodiments according to FIG. 3 and FIG. 4.

In these exemplary embodiments, it is, furthermore, provided to use the electrical conductibility of the treatment fluid flowing in the media cannula 18 for forming one of the electrodes of the circuit. In this case, the media cannula 18 is integrated in the conductor element 4 configured as a diamond electrode 26, so that a particularly compact construction can be achieved. The inner space of the media cannula 18 is, for its part, connected to the connection cable 8 and electrically connected via the latter to one of the poles of the electrical supply unit 10. In this way, the outlet opening 20 of the media cannula 18 forms from an electrical point of view a contact or electrical contact point, through which the current flows into the component in need of treatment. If the media cannula 18 and its outlet opening 20 are suitably positioned, if possible in the immediate vicinity of the component in need of treatment, and by using the outlet opening 20 as an electrical contact, it is achieved that the electric current applied for treatment and cleaning purposes can flow through the surface zone colonized by the bacteria of the component in need of treatment and from there, substantially directly, i.e. in particular without “detours” through further body tissue or the like, to the outlet opening 20 serving as a contact surface. The media cannula 18 including the electrically conductive treatment fluid flowing therein, and the corresponding connection elements thus forms in the exemplary embodiment the second conductor element forming an electrical current path towards the outlet opening 20.

It can be seen in the enlarged representation of FIG. 3 that the conductor element 4, which is configured in the region of its free end 14 as a diamond electrode 26, is designed for a localized positioning according to requirements, in the immediate vicinity of the component in need of treatment, so that the production of the hydroxyl radicals and H⁺ ions provided during treatment can take place in the immediate vicinity of the place of use. In the exemplary embodiment, this is explained by means of a dental implant 30 inserted into a patient's jaw bone; however, of course, other applications are also imaginable, in which a component, for example a bone implant of any type of construction, shall be cleaned of a contamination with a biofilm in a flexible and focused manner. FIG. 3 also shows a spatially limited spatial region 34 in the jaw bone 36, which is adjacent to the dental implant 30 in the region of its external thread 32 and which is affected by periimplantitis and, correspondingly, charged with bacteria.

It is in general a problem of dental-implant systems, in particular also of two-part implant systems, that inflammations or inflammatory foci may develop through a penetration of bacteria or germs into the tissue region near the place of insertion, in particular in the region of the external thread 32 worked into the jaw. Such inflammations, arising in particular also as a consequence of a so-called periimplantitis may lead to a serious degradation of the tissue and bone in the region of the place of insertion, in particular if they can develop and harden over a longer period of time. If no suitable counter-measures are taken, these degradations may cause a removal of the entire implant system from the bone and a replacement by other prosthetics. This most undesirable effect caused by the periimplantitis may, therefore, lead to a total loss of the implant system, so that renewed surgical measures, such as, for example, a scraping of the affected region in the jaw bone and the insertion of a new implant system, may become necessary. Such a removal may, furthermore, lead to a loss of bone or another loss of tissue substance, which in the extreme case may make it impossible to treat the patient with another implant. Such a new treatment necessitated by periimplantitis may become necessary even after relatively long periods of time after the first insertion of the implant system of, for example, up to some years or even decades.

The germs or bacteria observed in connection with a periimplantitis can in general colonize the inside of the components of the dental implant 30, but as a rule adhere preferably directly on the surface of the dental implant 30 inserted into the jaw bone 36 in the region of contact with the surrounding tissue or bone material, i.e. in particular in the region of the external thread 32. In the latter's region, the surface of the dental implant 30 can be provided with a roughening or the like, to particularly promote the growing-in into the tissue or the bone and to assist the healing-in of the dental implant 30 after its insertion. Especially in the region of such a roughening of the surface, which is actually considered as being particularly favorable for the implant system, a colonization of the germs or bacteria can increasingly take place, the roughness additionally impeding a specific removal of the existing germs or bacteria.

Therefore, suitable counter-measures are urgently desired, in order to be able, in case of a beginning or already existing periimplantitis or mucositis, to efficiently combat the inflammatory focus and kill the penetrated germs, preserving the implant system already inserted, so that afterwards, sound tissue or sound bone substance can form again in the region around the external thread 32. It is, therefore, desirable, in addition to a specific killing of the germs or bacteria in the region affected, to reliably remove also their material residues and fragments, possibly also the extracellular matrix and enotoxins, from the spatial region affected, so that then, the region affected can fill again with sound tissue or bone material and an intimate connection between the external surface of the dental implant 30 and the surrounding tissue or bone material can form again. In addition, the biofilm formed by the colonization of bacteria including the organic residues of killed bacteria should reliably be removed.

The treatment system 1 is provided for this purpose, i.e. for killing germs or bacteria in the region of insertion of the dental implant 30 and in particular also for subsequent flushing, removing and outputting the residues of tissue and material of the killed bacteria. With regard to its configuration and basic implementation, the treatment system 1 is based on two basic concepts, each of which is considered as independently inventive: On the one hand, it is designed for specifically killing the germs or bacteria existing in the region of insertion of the dental implant 30, through local generation and subsequent specific supply of a cleaning or disinfecting means, which is bacteriozide, but acceptable to the human organism. On the other hand, it is designed for detaching any residues or fragments of germs and/or bacteria still adhering on the surface of the dental implant 30, in particular in the region of the external thread 32, from the external surface of the dental implant 30 by a suitable application of current or current pulses, so that they can then be washed away.

In an aspect which is considered as independently inventive, both with regard to the configuration of the system and with regard to the provided process steps of the treatment method, the treatment system 1 is, therefore, designed, both structurally and functionally/conceptionally, for generating the ions or radicals provided for killing the germs or bacteria and/or cleaning the inserted implant part in a specific manner and only during the process control properly speaking, in the immediate vicinity of the region of insertion of the dental implant 30, in particular the region of the latter's external thread 32. This is effected by operating the zone of the end region of the conductor element 4 configured as a diamond electrode 26.

For detaching the bacteria or germs or their residues or fragments from the surface, it is provided to treat them through application of a direct current or alternatively or additionally with pulsed current pulses, because it has also turned out most surprisingly that it seems that especially this pulsed application of current pulses in combination with suitably chosen ion concentrations in the treatment fluid detaches the bacteria or germs or their fragments or residues from the surface below them in a particularly reliable manner, even if this surface is roughened and actually particularly promotes the adherence of organic material due to its surface structure.

In the embodiment represented in FIG. 3, the first conductor element 2, on the one hand, and the second conductor element 4 with the media cannula 18 integrated therein, on the other hand, are designed as components substantially independent of each other, which are electrically connected to the common supply unit 10. The conductor element 2 is configured, in the exemplary embodiment shown in FIG. 3, in the manner of a “conventional” electrode, i.e. in particular as an electrically conductive needle-shaped element made of metal. Due to this configuration, the conductor elements 2, 4 can be moved and positioned independently of each other, so that a particularly flexible treatment of the component in need of treatment is possible. In particular, the free ends 12, 14 provided in each case for contacting can be positioned on the component independently of each other and, therefore, possibly for an optimized electrical contact with the component.

Contrary thereto, the exemplary embodiment of FIG. 4 shows a variant, in which the diamond electrode 26, the conductor element 2 and possibly also the water supply, i.e. the media cannula 18, are integrated in a common housing 40. In the exemplary embodiment shown, the electrical conductors of the conductor element 2 and the diamond electrode 26 run coaxially to each other, the diamond electrode 26 concentrically surrounding the conductor element. The contact tip of the conductor element 2 can be approached up to the dental implant 30 and brought into contact therewith, for establishing the cathodic electrical contact. The outlet opening 20 of the media cannula 18 and thus the water supply can be accordingly positioned adjacent thereto. Usually, such an integrated construction with the common housing 40 for the media cannula 18 and the conductor element 2 enables an easier handling, because the person treating the patient can operate and position the system also with one hand only.

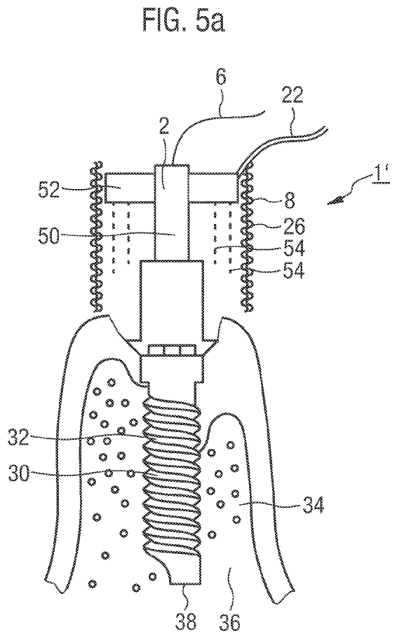

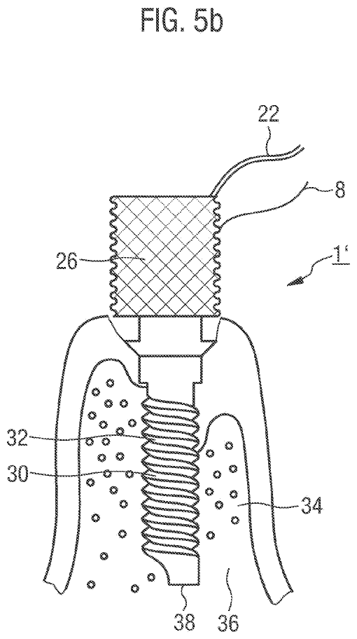

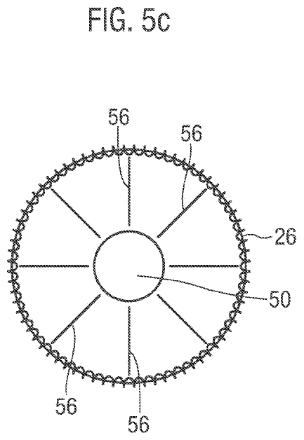

An alternative treatment system 1′, whose configuration is considered as independently inventive, is shown in FIG. 5 in an enlarged detail in a longitudinal section (FIG. 5a), in a lateral view (FIG. 5b) and in a cross-sectional view (FIG. 5c). This treatment system 1′, also referred to as “shower-head system” comprises as a first conductor element 2 a contact body 50 which is firmly connected with the dental implant 30 and which is connected via the connection cable 6 with the negative pole of the electrical supply unit 10 (not shown in detail in FIG. 5). The contact body 50 can be connected with the dental implant 30, for example, via a screw thread worked into the implant 30 or in any other suitable manner. Through the electrical contacting, the dental implant 30 is thus wired cathodically.

In an upper end region of the contact body 50, a distributor element 52 is arranged, which is connected via the connection hose 22 with the reservoir for a treatment fluid, in the exemplary embodiment, water. The distributor element 52 forms a kind of annular space around the upper end of the contact body 50, which can be flowed through by supplied water. Towards the bottom, this annular space of the distributor element 52 is perforated, so that the water held therein can escape downwards in droplets (comparable to the functioning of a shower head). This is indicated in FIG. 5a by the represented droplets 54.

The diamond electrode 26 is arranged in the manner of a concentric arrangement around the contact element 50 and in the latter's upper region around the distributor element 54, which in this embodiment is configured as a hollow cylinder. In order to provide a particularly large effective surface, the diamond electrode 26 is designed with a structured surface, for example as a grid or honeycomb structure. Just like in the other embodiments, the diamond electrode can be designed in particular on the basis of a metallic, ceramic or other suitably chosen base body, which is provided on its surface with a suitable, preferably boron-doped, diamond coating. The diamond electrode 26 is connected via the connection cable 8 to the positive pole of the electrical supply unit 10 and is thus wired anodically.

For further clarification, FIG. 5b shows the treatment system 1′ with a lateral view of the diamond electrode 26. The structured surface of the diamond electrode 26 can clearly be seen.

From the cross-sectional view of FIG. 5c, it can be seen that the contact element 50 is concentrically surrounded by the diamond electrode 26. Therebetween, a number of spacers 56 made of a suitably chosen insulating material are arranged. Furthermore, the diamond electrode 26 can be surrounded on its outside by a suitable insulating sheath.

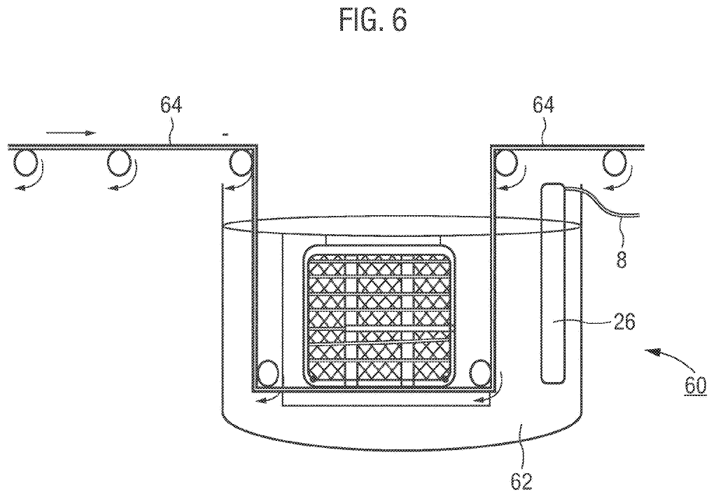

A very particularly preferred use, which is also considered as independently inventive, of a diamond electrode 26 for disinfecting or sterilizing a component, for example an implant intended to be inserted into the bone substance, or else osteosynthetic materials, such as, for example, plates or screws, or else medical or therapeutical instruments or tools, is shown in FIG. 6 by way of example for a sterilization system 60. The sterilization system 60 comprises a treatment bath 62, in which water or a hydrous fluid is available as a treatment fluid. The diamond electrode 26 is immersed in the treatment bath 62. The diamond electrode 26 is connected via the connection cable 8 to the positive pole of the electrical supply unit 10 and is thus wired anodically, analogously to the above-described variants.

A conveying belt 64 is guided through the treatment bath 62, which belt is suitably connected to the negative pole of the electrical supply unit and is thus wired cathodically. The components or instruments to be sterilized are transported on the conveying belt 64 and pass through the treatment bath 64. While passing, any biofilm or other contaminations present on the instruments or components is detached by means of a suitable energization, making use of the above-described process and the generation of the H⁺ ions taking place thereby, so that a sterilization can be achieved.

The particular advantage of such a configuration of the sterilization system 60 consists in particular in that a reliable disinfection or sterilization can take place in continuous operation and, therefore, with a relatively high throughput and treatment speed. As compared with conventional sterilization methods, for example for medical or dental instruments, considerably shorter treatment times can be achieved, entailing correspondingly considerable cost and logistics advantages.

### LIST OF REFERENCE NUMBERS

1 Treatment system

2, 4 Conductor element

6, 8 Connection cable

10 Supply unit

12, 14 End

16 Control unit

18 Media cannula

20 Outlet opening

22 Connection hose

24 Storage container

26 Diamond electrode

28 Clamp

29 Container

30 Dental implant

32 External thread

34 Spatial region

36 Jaw bone

38 Post part

40 Housing

42 End

50 Contact body

52 Distributor element

54 Droplet

56 Spacer

60 Sterilization system

62 Treatment bath

64 Conveying belt

What is claimed is:

1. A treatment method for cleaning bacterially contaminated surfaces of bone implants or dental implants (30), the method comprising:
a. providing at least two conductor elements (2, 4) connected to an electrical supply unit (10) for forming an electrical circuit,
b. positioning one of the conductor elements (2, 4) in electrical contact with a component in need of treatment,
c. positioning another of the conductor elements (2,4) in the immediate vicinity of the component in need of treatment, and
d. generating ions by applying an electrical current through the conductor elements (2, 4),
wherein the electrical current generates the ions via the decomposition of water molecules,
wherein the generated ions react to clean the component in need of treatment, and
wherein at least one of the conductor elements (2, 4) is designed as a diamond electrode (26).

2. The treatment method of claim 1, wherein the diamond electrode (26) is designed as a boron-doped diamond electrode (26).

3. The treatment method of claim 1, including providing a media cannula (18) and yielding a treatment fluid therefrom.

4. The treatment method of claim 2, including providing a media cannula (18) and yielding a treatment fluid therefrom.

5. The treatment method of claim 3, wherein the conductor element (2, 4) and the media cannula (18) are arranged in a common housing (40).

6. The treatment method of claim 4, wherein the inner space of the media cannula (18) is connected with a pole of the electrical supply unit (10) in an electrically conductive manner.

7. A treatment method for cleaning bacterially contaminated surfaces of bone implants or dental implants (30), the method comprising:
a. identifying a component in need of treatment, the component contaminated with a biofilms;
b. providing at least two conductor elements (2, 4) connected to an electrical supply unit (10) for forming an electrical circuit;
c. positioning one of the conductor elements (2,4) in electrical contact with the component in need of treatment; and
d. generating ions by applying an electrical current through the conductor elements (2, 4);
wherein the generated ions react to clean the component in need of treatment, and wherein at least one of the conductor elements (2, 4) is a diamond electrode (26).

8. The treatment method of claim 6, wherein the diamond electrode (26) is a boron-doped diamond electrode (26).

9. The treatment method of claim 6, wherein the method generates ions in the immediate vicinity of the component in need of treatment.

10. The treatment method of claim 6, wherein the method generates ions on the surface of the diamond electrode (26).

11. The treatment method of claim 6, wherein the method cleans the component in need of treatment, without an external supply of ions.

12. The treatment method of claim 6, wherein the electrical current generates the ions via the decomposition of water molecules.
